(19) United States
(12) Patent Application Publication
(10) Pub. No.: US 2011/0144006 A1
(43) Pub. Date: Jun. 16, 2011

(54) PROTEIN COMPOSITION

(75) Inventor: Stig Steen, Lund (SE)

(73) Assignee: Igelosa Life Science AB, Genarp (SE)

(21) Appl. No.: 12/638,502

(22) Filed: Dec. 15, 2009

(51) Int. Cl. A61K 38/16; A61P 3/04; A61P 3/10; A61P 3/00

(52) U.S. Cl. 514/4.8; 514/6.7; 424/451; 424/466

(57) ABSTRACT

The invention relates to a protein composition which upon ingestion by a healthy person with a normal fasting insulin level between 3.0-8.0 μU/L increases the insulin level to at most 20.0 μU/L. The invention may be in the shape of a powder, capsules, pellets, tablets, effervescent tablets or food products comprising said protein composition.

### FIELD OF INVENTION

The invention relates to a protein composition which upon ingestion by a healthy person, with a normal fasting insulin level between 3.0-8.0 μU/L, increases the insulin level to at most 20.0 μU/L. The invention may be in the shape of a powder, capsules, pellets, tablets, effervescent tablets or food products comprising said protein composition.

### BACKGROUND OF INVENTION

Sportsmen often ingest protein mixtures to facilitate muscle development and growth. One commonly used mixture is Triple Protein™, containing various preparations of whey protein (from milk). People with malnutrition, elderly people, patients with convalescence, or patients before an operation also benefit from an extra intake of dietary proteins. A high protein intake, including all the essential amino acids, is associated with a better health and a greater resistance against diseases of various kinds.

The human body regulates blood glucose (blood sugar) to be kept in a certain range. Insulin and glucagon are the hormones involved and insulin is secreted into the blood after a meal. Both insulin and glucagon are secreted from the pancreas, and thus are referred to as pancreatic endocrine hormones. The production of insulin by the pancreas is different in patients who suffer from diabetes, hypoglycemia, or other blood sugar problems.

The most full-worthy protein compositions that are available on the market today are mainly produced from whey. Such a protein composition will upon digestion give rise to an increase in the insulin level. Such an increase will stress the human body and the cells containing fat will be closed (keeping the fat inside the cells) which makes it hard to reduce body fat. It is commonly known that high insulin levels are also coupled to obesity and insulin resistance.

Therefore a study was initiated to develop and evaluate a protein mixture with similar amino acid content as in whey protein, but that eliminated the above defined problems of increasing the insulin response after a meal. Thereby it should be possible to solve the problems of supplying extra protein to people who for various reasons would benefit from that but are suffering from for example insulin resistance, obesity or the metabolic syndrome.

### SUMMARY OF THE INVENTION

The present invention relates to a protein composition for affecting the insulin levels in a subject comprising at least 76% (w/w) of proteins from at least one vegetable source and at least 0.2% (w/w) of at least one free amino acid. Said protein composition being an improved composition compared to other protein compositions available today being whey proteins in that the composition upon ingestion maintains the insulin levels at a low level. Thereby there will be no large insulin peaks in the human being ingesting said protein composition. The insulin level will not differ too much from the normal insulin level in the mammal, which ingests said protein composition. High insulin levels which are among other things coupled to obesity, insulin resistance as well metabolic syndrome.

The invention also relates to a pharmaceutical composition, capsules, tablets or effervescent tablets comprising said protein composition as well as food products comprising said protein composition and a method of treating a human suffering from a disorder or disease in which insulin is involved somehow, such as obesity, insulin resistance and metabolic syndrome. Additionally said protein composition give rise to a feeling of satisfaction upon ingestion, wherein said composition also is suitable to be used as a weight lowering product for humans. The invented product is also suitable to be used to reduce the fat mass and still maintaining the mass of muscles such as suitable for a body builder.

### BRIEF DESCRIPTION OF THE DRAWINGS

FIG. 1 shows the mean Plasma glucose (P-Glucose) response in six healthy individuals after an intake of 30 g of the invented protein mixture as compared to 30 g of a commercially available protein mixture based on whey (Triple Protein™). The P-glucose is negligible in both powder mixtures.

FIG. 2 shows the mean Serum insulin (S-Insulin) response in the same blood test and here the difference in insulin response is obvious.

### DETAILED DESCRIPTION OF THE INVENTION

### Definitions

“Vegetable source” can refer to any product that is generally referred by the public as a vegetable and can include artichoke, asparagus, beans (green, baked, pinto, black, soy etc.), beets, broccoli, Brussels sprouts, cabbage, carrot, cauliflower, celery, chick pea, corn, cucumber, eggplant, garlic, gourd, leek, lettuce, mustard, onion, peas, pepper, potato, pumpkin, spinach, squash, rice, lupin, turnips, yam, zucchini, and combinations and mixtures thereof. Specific vegetable sources may be obtained from seeds such as for example different kinds of potato, soybean, rice as well as bean.

Administering” a dosage or dosage form includes self-administration by the subject, administration by another to the subject, and providing advice for administration to the subject (as in instructions provided in a tangible medium, such as printed instructions or advice on a computer readable medium). Administration by another to the subject can include, for example, administration by a physician, nurse or other health care provider or dietary consultant (such as a nutritionist). Administration also includes providing an end product (such as a solid composition in tablet or powdered form or a liquid composition) that is consumed or utilized by the subject.

In the present context, amino acid names and atom names are used as defined by the Protein DataBank (PNB) (www.pdb.org), which is based on the IUPAC nomenclature (IUPAC Nomenclature and Symbolism for Amino Acids and Peptides (residue names, atom names etc.), Eur J. Biochem., 138, 9-37 (1984) together with their corrections in Eur J. Biochem., 152, 1 (1985). The term “amino acid” is intended to indicate an amino acid from the group consisting of alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W) and tyrosine (Tyr or Y).

A “free” or a “free-form” amino acid is not covalently bonded to any other amino acid, and thus is not included in a protein or polypeptide. A free amino acid can generally be absorbed into the bloodstream without degradation. Chemical derivatives of a free amino acid that can substitute for the amino acid in a biochemical process are also encompassed by this disclosure. Thus, the amino acids can be used in the form of metal salts such as sodium salt, potassium salt, inorganic acid salts such as hydrochloride, sulfate, or organic acid salts such as acetate, lactate, malate. There are cases in which the above-mentioned amino acids partly or wholly may be used in the form of N-acetyl derivatives, such as N-acetyl derivative and the like. The amounts of amino acids expressed herein are in the form of the free base, unless indicated otherwise in a specific example.

Amino acids can be present in two stereoisometric forms, called “D” and “L.” The D and L form of any amino acid have identical physical properties and chemical reactivities, but rotate the plane of plane-polarized light equally but in opposite directions and react at different rates with asymmetric reagents. Most enzymes acting upon amino acids have asymmetric binding sites and thus can discriminate between the D and L forms. All naturally occurring amino acids in proteins are in the L form, although D-amino acids are found in some living cells, such as in the cell walls of microorganisms.

A “dosage form” comprises any preparation, or combination of preparations, that provides a desired dosage. Hence a dosage form can include a single composition (such as a capsule or other ingestible preparation) or a combination of several different compositions (such as a powdered beverage mix, a tablet and/or a liquid supplement). A dosage form can “provide a daily dosage” in either a single unit dosage form (such as a tablet or a powder) or in multiple dosages taken at different times throughout a day. Hence a dosage form that includes multiple sub-dosage forms can provide the total daily dosage administered at different times during a day (for example twice or three times a day), and in different forms (for example as a liquid beverage and a tablet).

Percent by weight” is the weight of a single ingredient divided by the weight of the total amount of ingredients, expressed as a percentage. “Percent by weight of active ingredients” is the weight of a single active ingredient divided by the total weight of active ingredients. “Active” ingredients include amino acids as proteins. “Inactive” ingredients are those ingredients that do not have a therapeutic effect, such as solvents, carriers, preservatives and flavouring agents.

“Obesity” is a medical condition in which excess body fat has accumulated to the extent that it may have an adverse effect on health, leading to reduced life expectancy. Body mass index (BMI), which compares weight and height, is used to define a person as overweight (pre-obese) when their BMI is between 25 kg/m² and 30 kg/m² and obese when it is greater than 30 kg/m². Obesity is associated with many diseases, particularly heart disease, type 2 diabetes, breathing difficulties during sleep, certain types of cancer, and osteoarthritis. Obesity is most commonly caused by a combination of excessive dietary calories, lack of physical activity, and genetic susceptibility, though a limited number of cases are due solely to genetics, medical reasons or psychiatric illness. The primary treatment for obesity is dieting and physical exercise. If this fails, anti-obesity drugs may be taken to reduce appetite or inhibit fat absorption. In severe cases, surgery is performed or an intragastric balloon is placed to reduce stomach volume and or bowel length, leading to earlier satiation and reduced ability to absorb nutrients from food. Obesity is a leading preventable cause of death worldwide, with increasing prevalence in adults and children, and authorities view it as one of the most serious public health problems of the 21st century. Obesity is stigmatized in the modern Western world, though it has been perceived as a symbol of wealth and fertility at other times in history, and still is in many parts of Africa.

“Insulin resistance” is the condition in which normal amounts of insulin are inadequate to produce a normal insulin response from fat, muscle and liver cells. Insulin resistance in fat cells reduces the effects of insulin and results in elevated hydrolysis of stored triglycerides in the absence of measures which either increase insulin sensitivity or which provide additional insulin. Increased mobilization of stored lipids in these cells elevates free fatty acids in the blood plasma. Insulin resistance in muscle cells reduces glucose uptake (and so local storage of glucose as glycogen), whereas insulin resistance in liver cells results in impaired glycogen synthesis and a failure to suppress glucose production. Elevated blood fatty acid levels (associated with insulin resistance and diabetes mellitus Type 2), reduced muscle glucose uptake, and increased liver glucose production all contribute to elevated blood glucose levels. High plasma levels of insulin and glucose due to insulin resistance are of importance for the development of the symptoms of metabolic syndrome and type 2 diabetes, including its complications

“Metabolic syndrome” is a combination of medical symptoms that increase the risk of developing cardiovascular disease and diabetes. It affects one in five people, and prevalence increases with age. Some studies estimate the prevalence in the USA to be up to 25% of the population. Metabolic syndrome is also known as metabolic syndrome X, syndrome X, insulin resistance syndrome, Reaven's syndrome, and CHAOS (Australia). A similar condition in overweight horses is referred to as equine metabolic syndrome; it is unknown if they have the same etiology.

“Base level” is intended to be the insulin level that the human being has prior to ingestion of the protein compositions of the invention. The base level being dependent of the human being as well as what the human being has eaten prior to that the protein compositions of the invention are to be consumed. See the examples.

### Protein Composition

The invention relates to a protein composition wherein said proteins mainly are derived from vegetable sources, being optimal for human beings and which keeps the insulin levels low upon ingestion of said protein composition, which reduces or eliminates the development of disorders or diseases coupled to an increase in the insulin secretion as well as abnormal insulin peaks.

The protein composition is intended to be utilised for affecting the insulin levels in a subject and comprises at least 76% (w/v) of proteins from at least one vegetable source and at least 0.2% of at least one free amino acid, such as 77, 78, 79, 80, 81, 82, 83, 84, 85, 86, 87, 88, 89, 90, 91, 92, 93, 94, 95, 96, 97, 98 or 99% or even more proteins, at most 99.8%.

Normally the fast values for insulin vary between about 3.0 to about 8.0 μU/L in a human (being the base level). However, if the human suffers from any disease such as obesity of insulin resistance the values may be different. Upon ingestion of a whey protein composition the insulin levels will increase tremendous from the fast values and give rise to high peak values (see the examples).

The invented protein composition has been shown to give rise to lower insulin levels compared to the whey proteins. The invented protein composition, will normally upon ingestion increases the insulin levels to at most 20.0 μU/L in a healthy person with a normal fasting insulin level between 3.0-8.0 μU/L. Examples of increased levels includes 16.0, 17.0, 18.0, 19.0 μU/L. However, often the invented product will not give rise to higher insulin levels than about 10, 11, 12, 13, 14, 15, 16, 17, 18, 19 or 20 μU/L.

Insulin levels that will not stress the human body, stress that normally could result in the development of different disorders or diseases such as obesity, insulin resistance of the metabolic syndrome. Additionally, the invented protein composition upon ingestion maintains the glucose levels below 1 mmol/L compared to the base value, which in healthy persons normally ranges from about 3.5 to about 7 mmol/L.

The protein composition may be obtained from any kind of vegetable source such as spinaches, nuts, lupin, cauliflowers, broccolis, potatoes, rice, beans, and peas.

The nutrition powder may be obtained from one or more sources and may comprise a mixture of amino acid isolates and/or protein sources or isolates. For certain purposes the proteins are preferred due to that the proteins are more natural and the human being then needs to degrade the proteins by themselves which also for certain purposes improves the amino acid profile that a specific human being needs. The nutrition powder may mainly be from plant sources, which sources depend on what and who should use the nutrition powder. This also applies when the nutrition powder will be consumed by vegetarian people or people who prefer plants sources. The nutrition powder may also be a mixture of different sources, such as a mixture of at least two, three, four or more different plant sources. One specific example is when the nutrition powder is obtained from three or four different plant sources. By the use of proteins from plants, it is possible to prepare a nutrition powder which is free from casein as well as lactose and people being allergenic against those compounds can digest the powder without any risk.

The protein composition may be obtained from seeds, such as peas, potatoes, soy beans or the like. One example is that the proteins are obtained from 1, 2, 3, 4 or more vegetable sources. Optionally one or more free amino acids may be added to said protein composition such as 1, 2 3, 4, 5 or more free amino acids. If free amino acids are added to said protein composition it may be at least 0.2% (w/w) and at most 10% (w/w) of free amino acids, such as 1, 2, 3, 4, 5, 6, 7, 8 or 9%. Examples of free amino acids are Trp, Thr, Leu, Pro and Glu.

One example of a protein composition comprises; about 35 to about 45% of proteins from pea, such as 36, 39, 40 or 43%, about 23 to about 45% of proteins from potato, such as 28, 30, 35 or 40%, about 10 to about 15% of proteins from soy bean, such as 11, 12, 13 or 14% and about 0.2 to about 10% free amino acids, such as 1, 2, 3, 4, 5, 6, 7, 8 or 9%.

By the development of such a protein composition it is for the first time possible to maintain the insulin level at reasonable low levels during the ingestion of said protein composition, which has not been possible before due to that normally proteins from whey is used which gives rise to insulin peaks (see examples). This is in comparison with other protein compositions such as those shown in the examples.

The main part of the proteins may be substantially intact and the protein composition is substantially free from heavy metals as well as antibiotics.

If the protein composition is used as a food product additional components may be added to the protein composition to improve the nutritional value. Other examples are vitamins, minerals and omega fatty acids such as polyunsaturated fatty acids. Other components may be flavouring agents, aroma substances as well as sweetening agents. Examples of sweeteners are sucralose, sodium cyclamate, acesulfame K, neohesperidin dihydrochalcone, glycyrrhizin, steveoside, monellin, thaumatin, aspartame, dulcin, saccharin, naringin dihydrochalcone, neotame and a mixture of two or more thereof. Examples of other additives are aroma substances such as vanillin, dyes, taste substances, emulsifiers such as lecithin, thickeners such as pectin, carob bean meal, guar gum, antioxidants, preservatives, minerals such as sodium or calcium, in particular salts such as sodium chloride, roughage, fibers, triglycerides of medium chain lengths, phytoestrogens, natural or synthetic vitamins such as vitamin A, vitamin B.sub.1, vitamin B.sub.2, vitamin B.sub.3, vitamin B.sub.5, vitamin B.sub.6, vitamin B.sub.12, vitamin B complex, vitamin C, vitamin D, vitamin E, vitamin F and vitamin K, or combinations thereof.

A further additive to a food product is a probiotic microorganism, a probiotic. These are taken to mean, in the present case, a living microbial additional component which, by stabilizing or enhancing the microbial composition in the digestive tract of the human or animal consumer, promotes the health thereof. Such microorganisms which can be used, for example, in foods, are, for example: Bifidobacterium such as the strains B. adolescentis, B. animalis, B. bifidum, B. longum, B. thermophilum; Enterococcus; Lactobacillus, such as the strains Lb. acidophilus, Lb. brevis, Lb. casei, Lb. cellobiosus, Lb. crispatus, Lb. delbrueckii subscp. Bulgaricus, Lb. fermentum, Lb. GG, Lb. johnsonii, Lb. lactis, Lb. plantarum, Lb. reuteri, Lb. rhamnosus, Lb. salivarius; Bacillus cereus toyoi; Bacillus cereus; Leuconostoc; Pediococcus acidilactici; Propionibacterium; Streptococcus such as the strains S. cremoris, S. infantarius, S. intermedius, S. lactis, S. salivarius subsp. thermophilus (cf. Fuller (1989) J. Appl. Bacteriol.). Preferred probiotics are bacteria of the genera Lactobacillus and Bifidobacterium. Such probiotic bacterial cultures can preferably be implemented as dry cultures or long-life cultures.

A further additive is a symbiotic. In the present case these are taken to mean a mixture of at least one prebiotic and at least one probiotic which, by enhancement of the survival rate and increasing the number of health-promoting living microbial organisms in the gastrointestinal tract, promotes the health of the human or animal consumer, in particular by selective stimulation of the growth and/or metabolic activity of the microbial organisms.

Said protein composition may be formulated for oral administration, such as a capsule, tablet or effervescent tablet.

The protein composition may also be a powder or pellets. Pellets may be advantage if the protein composition will be transported over long distances or stored for a longer period in sachets such as those sachets that are used for conventional animal food products. The pellets may also be made in the same manner as conventional animal food products are produced using the same kind of equipment.

It is especially advantageous to formulate oral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect. The dosage unit as defined above may comprise from about 10 g of proteins and free amino acids, such as from about 10 to about 40 g of proteins and free amino acids.

### Tablets or Capsules Containing the Protein Composition

The above, mentioned composition may be in the form of a tablet depending on the user and the use.

For oral administration, tablets containing various excipients such as microcrystalline cellulose, sodium citrate, calcium carbonate, dicalcium phosphate and glycine may be employed along with various disintegrants such as starch (and preferably corn, potato or tapioca starch), alginic acid and certain complex silicates, together with granulation binders like polyvinylpyrrolidone, sucrose, gelatin and acacia. Additionally, lubricating agents such as magnesium stearate, sodium lauryl sulfate and talc are often very useful for tabletting purposes. Solid compositions of a similar type may also be employed as fillers in gelatin capsules; preferred materials in this connection also include lactose or milk sugar as well as high molecular weight polyethylene glycols. When aqueous suspensions and/or elixirs are desired for oral administration, the active ingredient may be combined with various sweetening or flavoring agents, coloring matter or dyes, and, if so desired, emulsifying and/or suspending agents as well, together with such diluents as water, ethanol, propylene glycol, glycerin and various like combinations thereof.

A composition for tableting or capsule filling may be prepared by wet granulation. In wet granulation, some or all of the active ingredients and excipients in powder form are blended and then further mixed in the presence of a liquid, typically water, which causes the powders to clump into granules. The granulate is screened and/or milled, dried and then screened and/or milled to the desired particle size. The granulate may then be tableted, or other excipients may be added prior to tableting, such as a glidant and/or a lubricant.

A tableting composition may be prepared conventionally by dry blending. For example, the blended composition of the actives and excipients may be compacted into a slug or a sheet and then comminuted into compacted granules. The compacted granules may subsequently be compressed into a tablet.

As an alternative to dry granulation, a blended composition may be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate and colloidal silica. The proper use of these and other excipients in direct compression tableting is known to those in the art with experience and skill in particular composition challenges of direct compression tableting.

A capsule filling of the present invention may comprise any of the aforementioned blends and granulates that were described with reference to tableting, however, they are not subjected to a final tableting step.

Effervescence is the reaction (in water) of acids and bases producing carbon dioxide. The proportion of acids may be varied, as long as the bicarbonate is completely neutralised.

Examples of acids used in this reaction are citric acid, tartaric acid, malic acid, fumaric acid, adipic acid, acid citrates, succinic acid and mixtures thereof. Citric acid is the most commonly used, and it imparts a citrus-like taste to the product. Examples of bases used in the effervescent reaction are sodium carbonate, potassium carbonate, sodium bicarbonate, potassium bicarbonate, calcium bicarbonate, magnesium carbonate, sodium glycocarbonate, carboxylysine and mixtures thereof. Sodium bicarbonate is very common in effervescent formulas.

The above mentioned composition may comprise at least one additive selected from the group comprising excipients, lubricants, emulgators, fillers, surfactants (e.g., polysorbate 80 and sodium lauryl sulfate), flavours and colours, including natural or synthetic ones, vitamins, sweeteners (acesulfame potassium, sodium saccharin, aspartame, and surcalose), nutritional additives (e.g antioxidants), and mixtures thereof.

Substances giving taste, colour or antioxidative properties to the composition can be plant polyphenols (Cheynier V. Am J Clin Nutr. 2005; 81: 223-229) coming from natural sources such as blueberries, cranberries, grapes and tea leaves.

The composition may include one or more excipient, such as a pharmaceutical excipient or a food additive. Examples of suitable excipients include, e.g., starches, natural gums, cellulose gums, microcrystalline cellulose, methylcellulose, cellulose ethers, sodium carboxymethylcellulose, ethylcellulose, gelatin, dextrose, lactose, sucrose, sorbitol, mannitol, polyethylene glycol, polyvinylpyrrolidone, pectins, alginates, polyacrylamides, polyvinyloxoazolidone, polyvinylalcohols and mixtures thereof.

Additionally the composition may contain various lubricants suitable for use in the composition including water dispersible, water soluble, water insoluble lubricants and combinations thereof. Examples of useful water soluble lubricants include sodium benzoate, polyethylene glycol, L-leucine, adipic acid, and combinations thereof.

The composition may also include water insoluble lubricants including, e.g., stearates (e.g., magnesium stearate, calcium stearate and zinc stearate), oils (e.g., mineral oil, hydrogenated and partially hydrogenated vegetable oils, and cotton seed oil) and combinations thereof. The effervescent part may also comprise vitamins, and minerals as disclosed in U.S. Pat. No. 4,725,427 “Effervescent vitamin-mineral granule preparation”. Effervescent energy tablet dissolving in any cold beverage (fruit juice, sports drinks, iced teas, sodas, etc.) to create an instant energy drink are commercially availed and such compositions may also be formulated according to the invention containing our invented composition.

The invention also relates to a food product comprising said protein composition as defined above.

The invention also relates to a method of treating a mammal having high insulin levels by administrating to said mammal a protein composition, or the powder, pellet, capsule or tablet or effervescent tablet or the dosage unit or the food product as defined above or in combination with other compounds and medicaments to for example a mammal that suffers from obesity, insulin resistance or metabolic syndrome.

### EXAMPLES

### Example 1

Protein powder from rice was purchased from RICE PROTEIN TECHNOLOGIES LLC under the trademark NovaPro™70, being a Rice protein concentrate from brown rice. A Soy Protein Isolate powder was purchased from REMY INDUSTRIES under the trademark Remypro N80+, Pea protein Isolate was purchased from COSUCRA GROUP under the trademark Pisane® and purified potato protein was purchased from EMSLAND-STÄRKE GmbH. By mixing these different protein powders and free amino acids in a specific relationship, a protein composition mixture with an amino acid profile similar to whey protein was obtained.

### Protein Composition 1:

1 kg mix based on potato, pea and soy protein:
Potato protein: 400 gram (40%)
Pea protein: 400 gram (40%)
Soy protein: 135 gram (13.5%)
25 L-Proline: 35 gram (3.5%)
L-Glutamin: 15 gram (1.5%)
L-Leucine: 10 gram (1.0%)
L-Tryptopan: 5 gram (0.5%)
Total: 1000 gram

### Protein Composition 2:

1 kg mix based on potato, pea, rice and soy protein:
Potato protein: 260 gram (26%)
Pea protein: 432 gram (43.2%)
Soy protein: 130 gram (13.0%)
Rice protein: 130 gram (13.0%)
L-Proline: 33 gram (3.3%)
L-Glutamin: 2 gram (0.2%)
L-Leucine: 7 gram (0.7%)
L-Tryptopan: 6 gram (0.6%)
Total: 1000 gram

### Example 2

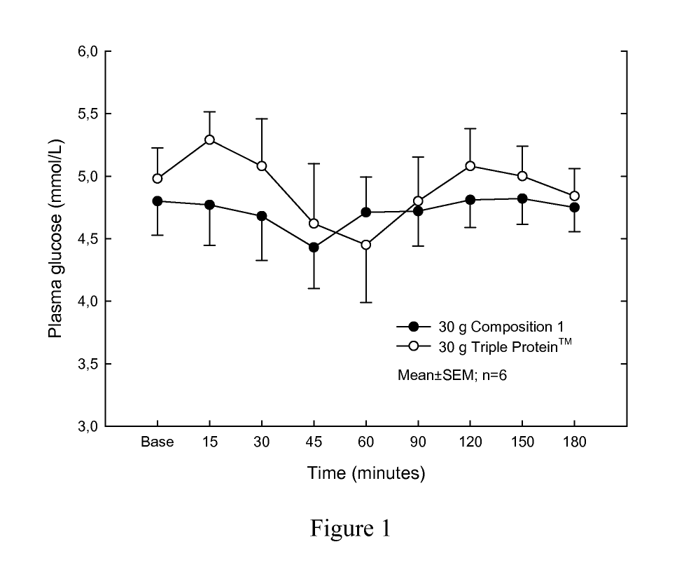

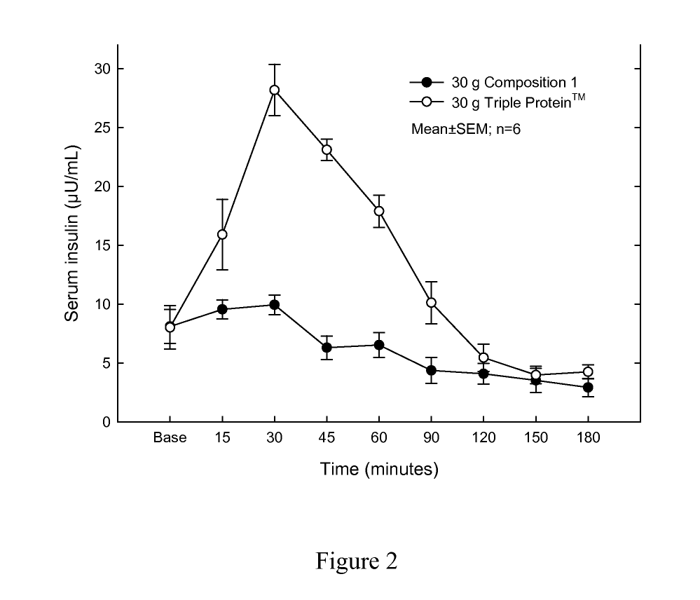

Four separate studies were performed (table 1-4). In table 1-2 six healthy individuals were recruited among the Igelösa Life Science research group. Gender and age are given in the various tables. Table 1 shows the P-glucose and S-insulin response from the intake of 30 g protein from the invented protein composition 1 (see Example 1). Table 2 shows the P-glucose and S-insulin response in the same test persons after an intake of 30 g protein from whey (TripleProtein™, containing ion-exchanged whey isolate, whey peptides as well as digested whey protein concentrate, wherein 84 gram protein is present in 100 gram of the product). Table 1-2 are also illustrated in FIG. 1-2.

Table 3-4 shows the P-glucose and S-insulin response from four of the same persons after an intake of 10 g respectively 40 g protein from the invented protein composition 2 (see Example 1).

Plasma glucose (P-glucose) was measured using absorption photometry (Cobas Integra 400, Roche Instrument Center, CH-6343 Rotkreutz, Schweiz). Serum insulin (S-insulin) was measured by Immunoassay (Roche Diagnostics Elecsys, Hitachi High Technologies Corporation, Tokyo, Japan). All the measurements were performed at the Igelösa Medical Clinic, which is an officially registered medical clinic in Sweden.

In all studies the participants had their normal breakfast before 7 am at home. The studies started at 10 am. A venflon was inserted in an anticubital vein and blood was withdrawn to obtain base values. The participants received different amounts of protein (see tables below). The drink was ingested from time 0 and during 12 minutes. After 15 minutes a blood sample was taken. Then blood samples were collected at 30, 45, 60, 90, 120, 150 and 180 minutes and immediately analysed. All the participants were sitting or performed easy work between the blood samples. No coffee or other meals were allowed during the test.

The P-glucose values below are in mmol/L and the S-insulin values in μU/mL.

TABLE 1
Plasma glucose (upper panel) and serum insulin (lower panel)
before (Base) and the first three hours after intake of 30
g protein from the invented composition 1 + 500 ml water.

| | | | | | | | |
|---|---|---|---|---|---|---|---|
| Time | Female | Male | Male | Male | Male | Male | Mean |
| (min) | 60 years | 59 years | 31 years | 45 years | 27 years | 28 years | value |

P-glucose (mmol/L)

| | | | | | | | |
|---|---|---|---|---|---|---|---|
| Base | 4.92 | 5.62 | 3.76 | 5.36 | 4.50 | 4.63 | 4.80 |
| 15 | 4.97 | 5.74 | 3.78 | 5.56 | 4.07 | 4.48 | 4.77 |
| 30 | 5.37 | 5.81 | 3.76 | 5.15 | 3.93 | 4.04 | 4.68 |
| 45 | 4.54 | 5.77 | 3.55 | 4.82 | 3.83 | 4.09 | 4.43 |
| 60 | 4.8 | 5.89 | 4.1 | 5.06 | 4.28 | 4.13 | 4.71 |
| 90 | 4.96 | 5.85 | 3.96 | 4.96 | 4.38 | 4.21 | 4.72 |
| 120 | 4.93 | 5.65 | 4.26 | 5.17 | 4.41 | 4.43 | 4.81 |
| 150 | 4.86 | 5.65 | 4.46 | 5.11 | 4.27 | 4.55 | 4.82 |
| 180 | 4.78 | 5.50 | 4.28 | 5.12 | 4.37 | 4.47 | 4.75 |

S-insulin (μU/mL)

| | | | | | | | |
|---|---|---|---|---|---|---|---|
| Base | 4.14 | 9.18 | 10.70 | 4.77 | 14.59 | 5.21 | 8.10 |
| 15 | 7.01 | 8.57 | 11.78 | 10.94 | 6.55 | 12.45 | 9.55 |
| 30 | 13.71 | 11.02 | 9.93 | 7.34 | 6.65 | 10.96 | 9.94 |
| 45 | 8.42 | 9.58 | 3.28 | 4.36 | 4.78 | 7.38 | 6.30 |
| 60 | 9.18 | 10.72 | 4.70 | 5.17 | 4.65 | 4.76 | 6.53 |
| 90 | 6.00 | 8.20 | 3.97 | 3.09 | 2.08 | 2.89 | 4.37 |
| 120 | 4.44 | 6.86 | 4.21 | 2.90 | 1.62 | 4.51 | 4.09 |
| 150 | 2.79 | 6.91 | 3.23 | 3.41 | 1.03 | 3.79 | 3.53 |
| 180 | 3.09 | 5.39 | 2.04 | 2.48 | 1.69 | 2.83 | 2.92 |

TABLE 2
Plasma glucose (upper panel) and serum insulin (lower panel)
before (Base) and the first three hours after intake of 30
g protein from Triple Protein ™ (whey) + 500 ml water.

| | | | | | | | |
|---|---|---|---|---|---|---|---|
| Time | Female | Male | Male | Male | Male | Male | Mean |
| (min) | 60 years | 59 years | 31 years | 45 years | 27 years | 28 years | value |

P-glucose (mmol/L)

| | | | | | | | |
|---|---|---|---|---|---|---|---|
| Base | 4.75 | 5.99 | 4.64 | 5.12 | 4.92 | 4.44 | 4.98 |
| 15 | 5.31 | 6.13 | 5.32 | 5.27 | 5.19 | 4.51 | 5.29 |
| 30 | 5.13 | 6.33 | 4.59 | 5.43 | 5.20 | 3.77 | 5.08 |
| 45 | 5.19 | 6.24 | 3.72 | 3.99 | 4.96 | 3.59 | 4.62 |
| 60 | 3.90 | 6.31 | 3.85 | 4.28 | 4.65 | 3.72 | 4.45 |
| 90 | 4.50 | 6.28 | 4.61 | 4.73 | 4.68 | 3.99 | 4.80 |
| 120 | 4.91 | 6.32 | 4.65 | 5.16 | 5.04 | 4.37 | 5.08 |
| 150 | 4.95 | 6.00 | 4.76 | 4.95 | 4.91 | 4.40 | 5.00 |
| 180 | 4.88 | 5.64 | 4.77 | 4.90 | 4.73 | 4.13 | 4.84 |

S-insulin (μU/mL)

| | | | | | | | |
|---|---|---|---|---|---|---|---|
| Base | 5.21 | 10.02 | 17.87 | 4.77 | 5.77 | 4.50 | 8.02 |
| 15 | 6.97 | 16.84 | 36.15 | 6.65 | 6.23 | 22.58 | 15.90 |
| 30 | 17.34 | 24.92 | 45.50 | 37.54 | 15.70 | 27.99 | 28.17 |
| 45 | 23.63 | 28.29 | 20.43 | 15.93 | 24.74 | 25.58 | 23.10 |
| 60 | 15.76 | 27.10 | 17.79 | 9.38 | 17.38 | 19.92 | 17.89 |
| 90 | 6.92 | 16.98 | 17.03 | 3.27 | 7.38 | 9.14 | 10.12 |
| 120 | 6.18 | 9.42 | 6.87 | 2.47 | 5.40 | 2.36 | 5.45 |
| 150 | 3.61 | 6.71 | 4.41 | 2.60 | 3.76 | 2.76 | 3.98 |
| 180 | 3.99 | 6.65 | 4.00 | 3.11 | 3.99 | 3.73 | 4.25 |

TABLE 3
Plasma glucose (upper panel) and serum insulin (lower panel)
before (Base) and the first two hours after intake of 10 g
protein from the invented composition 2 + 500 ml water.

| | | | | | |
|---|---|---|---|---|---|
| Time | Female | Female | Male | Male | Mean |
| (min) | 62 years | 60 years | 60 years | 32 years | value |

P-glucose (mmol/L)

| | | | | | |
|---|---|---|---|---|---|
| Base | 5.24 | 4.85 | 5.56 | 4.42 | 5.02 |
| 15 | 5.44 | 4.69 | 5.60 | 4.67 | 5.10 |
| 30 | 5.37 | 4.83 | 5.51 | 4.43 | 5.04 |
| 45 | 5.29 | 4.69 | 5.51 | 4.23 | 4.93 |
| 60 | 5.51 | 4.97 | 5.49 | 4.24 | 5.05 |
| 90 | 5.72 | 4.75 | 5.36 | 4.23 | 5.02 |
| 120 | 5.74 | 4.83 | 5.71 | 4.56 | 5.21 |

S-insulin (μU/mL)

| | | | | | |
|---|---|---|---|---|---|
| Base | 7.03 | 2.12 | 4.35 | 5.76 | 4.82 |
| 15 | 10.49 | 3.50 | 7.25 | 8.18 | 7.36 |
| 30 | 11.86 | 5.25 | 7.32 | 6.76 | 7.80 |
| 45 | 9.95 | 4.91 | 5.67 | 4.79 | 6.33 |
| 60 | 8.45 | 5.30 | 5.00 | 4.08 | 5.71 |
| 90 | 6.71 | 1.71 | 4.15 | 2.02 | 3.66 |
| 120 | 6.51 | 2.47 | 4.67 | 2.80 | 4.11 |

TABLE 4
Plasma glucose (upper panel) and serum insulin (lower panel)
before (Base) and the first two hours after intake of 40 g
protein from the invented composition 2 + 500 ml water.

| | | | | | |
|---|---|---|---|---|---|
| Time | Female | Female | Male | Male | Mean |
| (min) | 62 years | 60 years | 60 years | 32 years | value |

P-glucose (mmol/L)

| | | | | | |
|---|---|---|---|---|---|
| Base | 5.97 | 5.05 | 5.76 | 4.22 | 5.25 |
| 15 | 5.79 | 5.17 | 6.13 | 4.05 | 5.29 |
| 30 | 5.53 | 5.28 | 6.12 | 3.46 | 5.10 |
| 45 | 5.58 | 5.16 | 6.14 | 3.81 | 5.17 |
| 60 | 5.65 | 4.99 | 6.16 | 3.84 | 5.16 |
| 90 | 5.86 | 5.45 | 6.18 | 4.26 | 5.44 |
| 120 | 6.65 | 5.41 | 5.78 | 4.19 | 5.26 |

S-insulin (μU/mL)

| | | | | | |
|---|---|---|---|---|---|
| Base | 12.98 | 2.78 | 5.11 | 8.08 | 7.24 |
| 15 | 14.15 | 4.63 | 8.32 | 13.52 | 10.16 |
| 30 | 13.05 | 6.42 | 11.67 | 16.49 | 11.91 |
| 45 | 13.06 | 6.47 | 13.44 | 13.63 | 11.65 |
| 60 | 16.00 | 8.67 | 14.46 | 7.51 | 11.66 |
| 90 | 15.33 | 7.61 | 10.26 | 9.42 | 10.66 |
| 120 | 9.64 | 7.73 | 7.03 | 4.98 | 7.35 |

### RESULT AND DISCUSSION

Blood glucose and insulin after ingestion of the special protein compositions 1-2 (example 1) are given in the tables above. It can be seen that all the tested the protein sources including the TripleProtein™ had a negligible effect on P-glucose. The low carbohydrate content is of importance for the insignificant effect on blood glucose. On the other hand, Triple Protein™ caused a clear S-insulin response in all participants whereas the invented protein composition did not significantly affect S-insulin. The down sloping S-insulin in some individuals is explained by the late postprandial response of the breakfast.

### CONCLUSION

A special vegetable protein composition based on potatoes, peas, rice and soy and five pure amino acids was tested. The mixture caused a minimal insulin response in contrast to another commonly used mixture based on whey protein (Triple Protein™). All protein sources had negligible effects on blood glucose.

1. A protein composition for affecting the insulin levels in a subject comprising at least 76% (w/v) of proteins from at least one vegetable source and at least 0.2% of at least one free amino acid.

2. The protein composition according to claim 1, wherein said composition comprises at most 10% free amino acids.

3. The protein composition according to claim 1, wherein said composition upon ingestion increases the insulin level to at most 20.0 μU/L in a healthy person with a normal fasting insulin level between 3.0-8.0 μU/L.

4. The protein composition according to claim 1, wherein said composition upon ingestion maintains the glucose levels below 1 mmol/L compared to the base line.

5. The protein composition according to claim 1, wherein said vegetable sources is obtained from seeds.

6. The protein composition according to claim 1, wherein said vegetable source is selected from the group consisting of spinaches, nuts, cauliflowers, broccolis, potatoes, lupin, rice, beans, and peas.

7. The protein composition according to claim 1, wherein said proteins are obtained from at least three vegetable sources.

8. The protein composition according to claim 1, wherein said proteins are obtained from pea, potato and soy bean or the like.

9. The composition according to claim 1, wherein said composition comprises at least one amino acid selected from the group consisting of Trp, Thr, Leu, Pro and Glu.

10. The protein composition according to claim 1, wherein said proteins are substantially intact.

11. The protein composition according to claim 1, wherein said composition comprises one or more flavouring agents or sweetening agents.

12. The protein composition according to claim 1, comprising at least one of vitamins, minerals and omega fatty acids.

13. The protein composition according to claim 1, wherein said composition is formulated for oral administration.

14. A powder, pellet, capsule or tablet or effervescent tablet comprising the protein composition according to claim 1.

15. A dosage unit comprising the protein composition according to claim 1, comprising from about 10 g of proteins and free amino acids.

16. The dosage unit according to claim 15, comprising from about 10 to about 40 g of proteins and free amino acids.

17. A food product comprising said protein composition according claim 1.

18. A method of treating a mammal having high insulin levels by administrating to said mammal a protein composition according to claim 1 or the powder, pellet, capsule or tablet or effervescent tablet or the dosage unit or the food product alone or in combination with other compounds and medicaments.

19. The method according to claim 18, wherein said mammal suffers from obesity, insulin resistance or metabolic syndrome.
